(19) United States
(12) Patent Application Publication
(10) Pub. No.: US 2009/0228012 A1
(43) Pub. Date: Sep. 10, 2009

(54) SURGICAL BORING TOOL SET

(75) Inventors: Valerie Gangji, Brussel (BE); Jean-Philippe Hauzeur, Brussel (BE)

Correspondence Address: KNOBBE MARTENS OLSON & BEAR LLP, 2040 MAIN STREET, FOURTEENTH FLOOR, IRVINE, CA, 92614, US

(73) Assignee: Universite Libre de Bruxelles, Brussel (BE)

(21) Appl. No.: 12/278,567

(22) PCT Filed: Feb. 16, 2006
(86) PCT No.: PCT/EP2006/001426

§ 371 Date: Sep. 12, 2008

(51) Int. Cl. A61B 17/00; A61B 17/58

(52) U.S. Cl. 606/80; 606/96

(57) ABSTRACT

A surgical device (1) is disclosed for making a bore in a tissue or a joint. The device includes an external trephine (2) having an open distal end (6) provided with a cutting rim (9), an open proximal end (7) and a hollow tubular portion (8). An internal trephine (3) is disclosed which is adapted to be positioned and to slide inside the external trephine (2), which has an open distal end (10) which is provided with a cutting rim (13), an open proximal end (11), and a hollow tubular portion (12). A guiding rod (4) is adapted to be positioned and to slide inside the internal trephine (3). The surgical device may also include an awl (5) which slides inside the external trephine (2) and has a distal end (18) provided with a cutting blade (38), and a proximal end (19) provided with a handle (21) and a solid tubular portion (20). A syringe (32) may be coupled to the internal (3) and/or external (2) trephine so that the surgical device can be also operated as an injection device. The device is useful for making a bore in a tissue or joint and/or for delivering a medicament or material to a tissue or joint.

### FIELD OF THE INVENTION

The present invention relates to the field of medicine. More in particular, the present invention relates to the field of medical instruments, particularly to surgical instruments. The present invention provides a surgical device which is adapted for making a bore or cavity in a tissue or joint. The present invention further provides a device which is adapted for delivering a medicament or material to a bore made in a tissue or joint. The present invention also relates to a method for making a bore in a tissue or joint and to a method for delivering a medicament or material to said bore in said tissue or joint.

### BACKGROUND

Bone diseases affect women, men, and children of all ages. From infancy to old age, bone diseases profoundly alter the quality of life.

An important bone disease includes osteonecrosis. This disease has been known by a number of other names including ischemic necrosis of bone, aseptic necrosis or avascular necrosis (AVN). Bone is a living tissue with living cells and a blood supply. Osteonecrosis (ON) refers to the death of bone which can occur by hypoxia from a decrease or loss in blood supply to specific parts of bones or by some other means. Eventually the dead section of bone weakens and collapses. There are two major forms of ON: post-traumatic and non-traumatic. Examples of post-traumatic ON include fractures and joint dislocations. Certain kinds of fracture, where the arterial blood vessels to a part of the bone have been interrupted or physically damaged, may result in ON. ON is called non traumatic when ON occurs without any history of trauma. Osteonecrosis most commonly occurs in large joints such as the hips, knees, ankles and shoulders. Pain, both at rest and, in particular, with movement of the joint and functional disability are the major symptoms of ON. Other symptoms include limitation of motion, joint stiffness and muscle spasms. Symptoms usually begin slowly. If the condition is left untreated, progressive bone damage and even bone collapse may occur, which can be associated with an increase in pain and loss of function of the joint.

Another important bone disease is osteoporosis which is characterized by a decreased bone density, with a loss of biomechanical strength and has a high prevalence and a high incidence of fractures. Osteoporosis and related fractures are particularly common in postmenopausal women; patients treated with corticosteroids m older individuals. It contributes substantially to the healthcare costs and burden of illness.

In certain types of fractures—so called non-union fractures or pseudarthrosis—a lack of, or a delayed bone healing may occur. The origin hereof could be at least partly due to a lack of proliferation of the osteoblastic cells.

Medical treatment of these diseases may include non weight bearing, bed rest, orthotics, and the administration of medication. In the treatment of above-named diseases also surgery may sometimes be required.

For ON, surgical techniques include included decompression of the necrotic area by taking of a plug of bone out of a diseased area and may involve creating a hole or bore in the bone or the joint. Another surgical technique may include bone grafting, which involves surgically removal of the dead bone and filling the empty space with bone graft that is either taken from the patient or from the bone bank.

Yet another therapeutic approach may also comprise cellular therapy. Such therapy involves the use of living cells which may be injected in cavities or bores applied in bone tissues or joints. Cellular material applied to the bone is used to replace, repair or ameliorate the biological function of the bone or joint tissue.

In the above-mentioned surgical techniques generally use is made of a surgical device comprising a trephine, which is applied to cut out discs of bone. Different kinds of such surgical devices have been described in the prior art.

For instance, U.S. Pat. No. 6,200,319 discloses a surgical trephine that is adapted for boring an opening in tamped bone chips which includes a hollow elongate body portion having an internal bore provided with a distal end which has an annular cutting rim. A hollow piston may be positioned in the bore. With this device an elongate cavity can be cut in bone chips. The debris or bone fragments resulting from the cutting enter the distal end of the internal bore and slowly push the piston up the bore away from the distal end. When the hollow elongate body portion is withdrawn from the cavity, the chips can then be easily dislodged from the bore by displacing the piston.

In another example U.S. Pat. No. 6,942,699 describes a bone recuperating surgical device comprising a trephine designed to be driven in rotation, open at both ends, provided with teeth on the periphery of its front opening and internally equipped with a bone crushing blade. In operation, the bone sample taken by the trephine is crushed by the bone crushing blade and fills the trephine. Subsequently, the crushed bone material may be injected in bone tissue by coupling the trephine to a bone injection syringe.

A problem with currently applied surgical devices, such as those enumerated above, is that the devices are generally not adapted for proper delivery of a (liquid) medicament or material in a bore made in the bone tissue or joint. A common problem encountered when delivering a liquid or material to an excavated tissue or joint when using a prior art surgical device is that the liquid material flows back out of the hole around the surgical device not only resulting in a considerably loss of material but also in possible and undesired liquid or material infiltration into the soft tissues around the bore.

Another drawback of currently known surgical devices is that the force that must be applied on the devices to introduce these in bone tissue is sometimes so high that the devices may bent or break during introduction. This does not only hamper proper surgical operation but may also result in substantial damage to tissue or joint.

Yet another problem is that the present surgical devices are not always optimally configured to fully reach treatment sites or to make bores of optimal dimensions, such that application of the surgical devices and delivery of a medicament or material may in some cases by technically challenging, difficult to perform with either manual or powered instruments, and destroying substantial tissue.

In view of the above, it is clear that there remains a need in the art for developing improved surgical devices for making a bore in a tissue or joint and for optionally delivering a medicament or material to such bore.

It is therefore an object of the present invention to provide an improved surgical device which overcomes at least some of the above-mentioned problems. More in particular, it is an object of the present invention to provide an improved surgical device which can be used for making a bore into a tissue or a joint, which is easier to handle, and which can be used for delivering a certain medicament or material in a tissue or joint in a safer and more accurate manner.

### SUMMARY

The present invention provides a hand-held surgical device adapted for making a bore in a tissue or a joint. The present invention also provides a surgical device which is particularly suitable for delivering a—preferably liquid—medicament, a material to said tissue or joint.

More in particular, in a first aspect the present invention provides a surgical device for making a bore in a tissue, such as a bone tissue, or in a joint, which comprises:
an external trephine having an open distal end provided with a cutting rim, an open proximal end and a hollow tubular portion,
an internal trephine, adapted to be positioned and to slide inside said external trephine, and having an open distal end provided with a cutting rim, an open proximal end, and a hollow tubular portion, and
a guiding rod, which is adapted to be positioned and to slide inside said internal trephine.

In another embodiment, the present invention provides a surgical device which further comprises an awl for penetrating a tissue or joint and which is adapted to be positioned and to slide inside said external trephine having a distal end provided with a cutting blade, a proximal end provided with a handle and a solid tubular portion. The present awl is particularly adapted to position the external trephine at a right place in the tissue or joint. Preferably, the cutting blade of the awl is provided with at least two, and preferably at least three cutting edges, and said cutting blade is particularly suitable for making a core biopsy, if necessary.

In a further embodiment, the invention provides a surgical device wherein the cutting rim of the external and of the internal trephine is provided with cutting teeth.

Preferably the teeth are curved. The configuration of the teeth of the internal and external trephines greatly improves clamping and penetration of the trephines in the tissue or joint, allowing making bores with neat edges and optimal dimensions. In addition, in another embodiment, the invention provides a surgical device wherein the distal end of the internal trephine has a conical shape. Preferably the teeth of the internal trephine extend to the interior of said external or internal trephine. The configuration of the teeth and of the distal end of the internal trephine greatly facilitates core biopsy, for instance to obtain bone marrow together with bone fibers.

In another embodiment, the invention provides a surgical device wherein the distal end of the external and/or internal trephine is provided with one or more holes. In yet another embodiment the distal end of the external and/or internal trephine may be provided with retractable spines. Such holes greatly facilitate application of a medicament or a material to bore that has been made in the bone tissue or joint. In addition, the retractable spines can advantageously be used to create micro-cavities or cracks into the surrounding bone tissue or joint tissues for facilitating the diffusion of a medicament or (cellular or synthetic) material applied to the bone tissue or joint.

In another further embodiment, the invention provides a surgical device wherein the proximal end of the external trephine is provided with an adaptor element comprising laterally extending wings for facilitating manual boring of the trephine in a tissue or joint.

In another preferred embodiment, the adaptor element of the external trephine further comprises a proximal abutment surface positioned proximally of the wings, which may be adapted to connect a syringe, such that the external trephine can be operated as an injection device.

In another further embodiment, the present invention provides a surgical device, wherein the proximal end of the internal trephine is also provided with an adaptor element comprising laterally extending wings for facilitating manual boring of the trephine in a tissue or joint. In yet another preferred embodiment, the adaptor element of the internal trephine further comprises a distal abutment surface positioned distally of the wings for limiting movement of the internal trephine in distal direction when inserted in the external trephine. Optionally, the adaptor element of the internal trephine further comprises a proximal abutment surface positioned proximally of the wings, which may be adapted to connect a syringe such that the internal trephine can be operated as an injection device.

In a further embodiment, the invention provides a surgical device, wherein the guiding rod comprises a solid tubular portion of which the proximal end is provided with an abutment surface for limiting movement of the guiding rod in distal direction when inserted in the external and/or internal trephine. Such rod greatly facilitates progression of the trephines in bone tissue. Furthermore, guiding rod of the present surgical device has dimensions and a configuration such that it is adapted to fit in the internal and/or external trephine. Using this guiding is particularly advantageous since it minimizes the risks of torsion and breaking of the internal and/or external trephine of the present device at the time of their introduction into a tissue or joint.

In another further embodiment, the invention provides a surgical device, wherein the length of the internal trephine is greater than the length of the external trephine, and wherein an extended portion of the internal trephine projects beyond the proximal end of the external trephine, when the internal trephine is fully inserted in the external trephine. Furthermore, the surgical device of the present invention is also characterized in that the length of the guiding rod is greater than the length of the internal trephine, and in that an extended portion of the guiding rod projects beyond the proximal end of the internal trephine, when the guiding rod is fully inserted in the internal trephine. In addition, the length of the awl is preferably greater than the length of the external trephine, and an extended portion of the awl projects beyond the proximal end of the external trephine, when the awl is fully inserted in the external trephine.

In a particularly preferred embodiment, the invention provides a surgical device wherein the outer diameter of the external trephine is smaller than 10 mm. In another preferred embodiment, the invention provides a surgical device wherein the outer diameter of the internal trephine is smaller than 10 mm. In addition, the awl has a diameter which is smaller than the inner diameter of the external trephine. Furthermore, the guiding rod has a diameter which is smaller than the inner diameter of the internal trephine. In a preferred embodiment, the internal trephine has an outer diameter which is preferably smaller than the inner diameter of the external trephine. The configuration of the internal trephine, in conjunction with the conical shape of its distal end of the internal external trephine advantageously enable to obtain bores with neat edges and optimal dimensions, and to take a core biopsy, if required, in an easy and clean way.

Another important advantage of the present invention is that the external and internal trephines of the present surgical device have a relatively small diameter. The small diameters of the trephines make it possible to penetrate in osseous tissue by small openings corresponding to these diameters. Trephines having such small diameters are particularly suitable for reaching tissues or joints in a subject's body while limiting tissue damage. In addition, the relatively small diameter of the trephine advantageously limits bone brittleness at the time of the passage of the surgical device in the bone and therefore also reduces secondary risks of fractures.

Using the present trephines, bores having small diameter can be made, which enables to deliver—preferably liquid or paste-like—medicaments or materials in the excavated osseous tissues or joints, in a small opening while limiting out-flow and thus loss of the delivered medicament or material.

In another aspect, the present invention further relates to a kit comprising a surgical device according to the invention comprising at least one internal trephine, external trephine, guiding rod, and optionally an awl, and a syringe for injecting a medicament or a material through said surgical device in a tissue or joint. The syringe is preferably adapted to be mounted on the proximal end of the internal and/or external trephine of the surgical device. In a preferred embodiment the syringe is pre-filled with a medicament or a material to be delivered to the tissue or joint. The present surgical device thus preferably comprises a set of different instruments, including an internal and an external trephine, a guiding rod and optionally an awl. The present set may comprise more than one internal and/or external trephine. For instance, the present kit may comprise an internal adapted for making a core biopsy, and an additional trephine adapted to be coupled to a syringe for injection of a medicament or a material in the bore made in the tissue or joint.

The present invention further relates in another aspect to the use of a surgical device according to the invention, or of a kit according to the invention, for making a bore in a tissue, such as a bone tissue, or in a joint. In another aspect, the present invention relates to the use of a surgical device according to the invention, or of a kit according to the invention, for delivering a medicament or material to a tissue, such as a bone tissue, or to a joint. The present surgical device and the present kit are particularly adapted for making bores and/or for treating a great number of local as well as systemic bone diseases or osseous defects by delivering medicaments, synthetic or cellular materials in diseased tissues or joints. More in particular, the present device and kit are particularly suitable for being used in cellular therapy of osseous diseases, i.e. for the delivery of cellular material in an excavated bore.

The present invention thus relates to an improved surgical instrument and a kit, which facilitates the formation of a bore or cavity in a tissue or joint, and which improves the introduction of one or more medicaments or materials in tissues or joints of a subject. More in particular, the present surgical device enables a bore to be formed quickly and with minimal trauma to the tissue or joint and minimal discomfort to the subject. The cutting rim of the trephines of the present device assures that the outer wall of the bore is cut cleanly, and improves boring. This results in a shorter boring period and a more accurately dimensioned bore. In addition, the present invention provides a device and a kit which may be used to assure a proper delivery without re-flow of a medicament or material.

With the insight to better show the characteristics and advantages of the present invention, some preferred embodiments and examples are described hereafter referring to the enclosed figures.

### DETAILED DESCRIPTION OF THE FIGURES

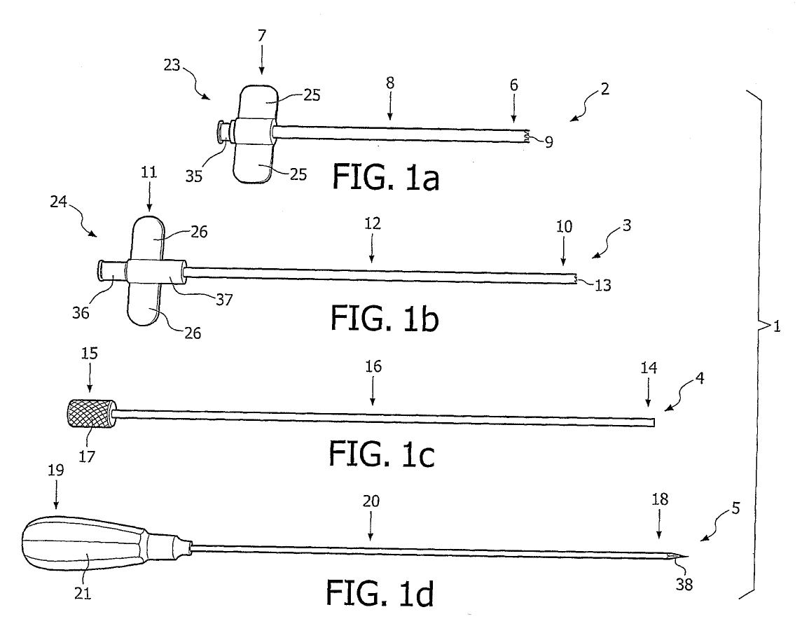

FIG. 1a represents an embodiment of an external trephine of a hand-held surgical instrument according to the present invention. FIG. 1b represents an embodiment of an internal trephine of a hand-held surgical instrument according to the present invention. FIG. 1c represents an embodiment of a guiding rod of a hand-held surgical instrument according to the present invention. FIG. 1d represents an embodiment of an awl of a hand-held surgical instrument according to the present invention.

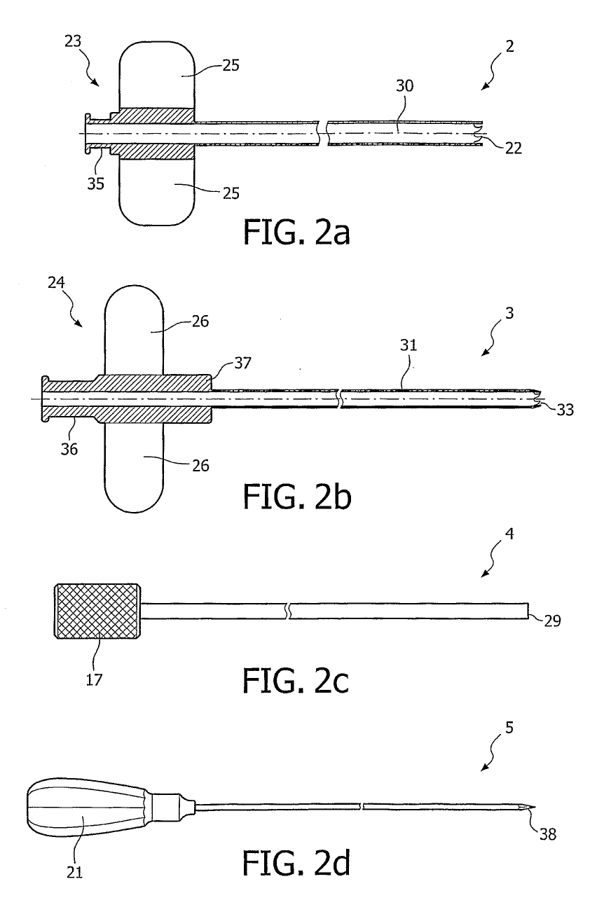

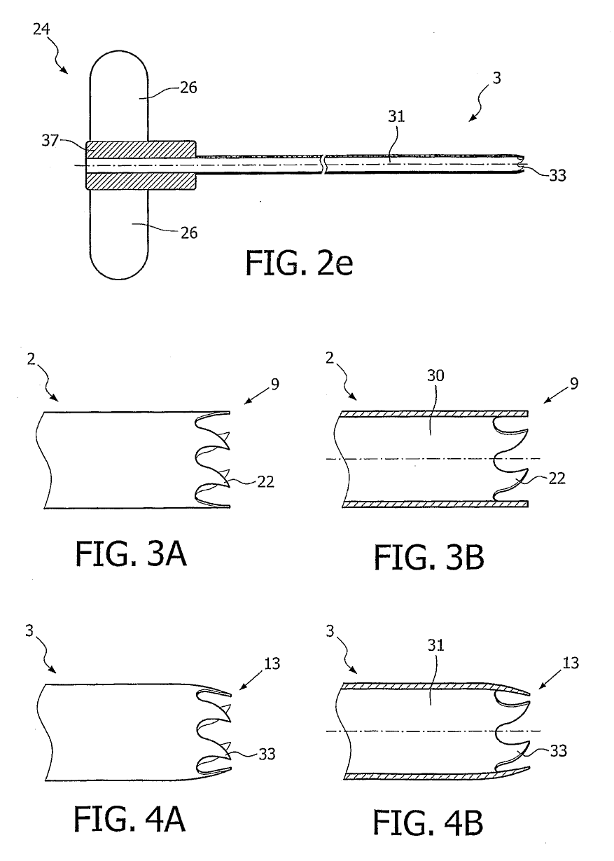

FIG. 2a-b is a cross-sectional view of the embodiments the elements represented in FIG. 1a-b. FIG. 2c represents another embodiment of a guiding rod of a hand-held surgical instrument according to the present invention. FIG. 2d represents another embodiment of an awl of a hand-held surgical instrument according to the present invention. FIG. 2e illustrates another embodiment of an internal trephine of a hand-held surgical instrument according to the present invention.

FIG. 3A illustrates a side view of the distal end of an embodiment of an external trephine according to the present invention. FIG. 3B is a cross-sectional view of the distal end of an embodiment of an external trephine according to the present invention.

FIG. 4A illustrates a side view of the distal end of an embodiment of an internal trephine according to the present invention. FIG. 4B is a cross-sectional view of the distal end of an embodiment of an internal trephine according to the present invention.

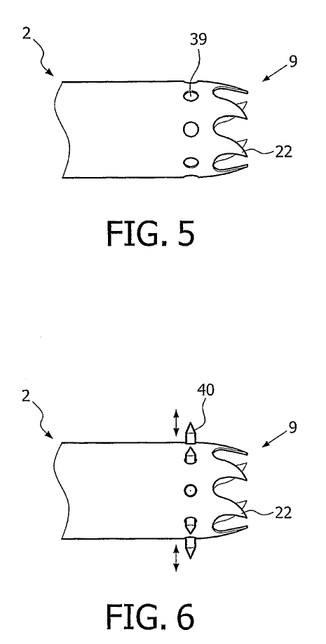

FIGS. 5 and 6 illustrates a side view of the distal end of other embodiments of an internal trephine according to the present invention.

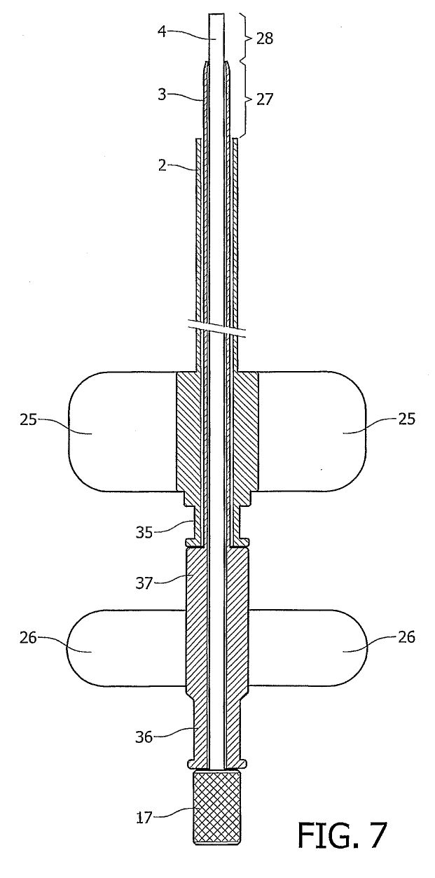

FIG. 7 is a cross-sectional view of an embodiment of a surgical device according to the present invention whereby different elements as represented in FIG. 1a-c of the device have been arranged with respect to each other.

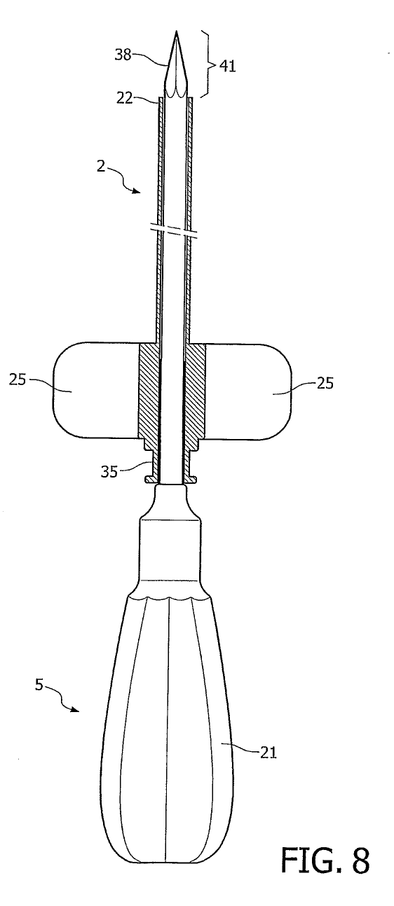

FIG. 8 is a cross-sectional view of an embodiment of a surgical device according to the present invention whereby different elements as represented in FIG. 1a and 1d of the device have been arranged with respect to each other.

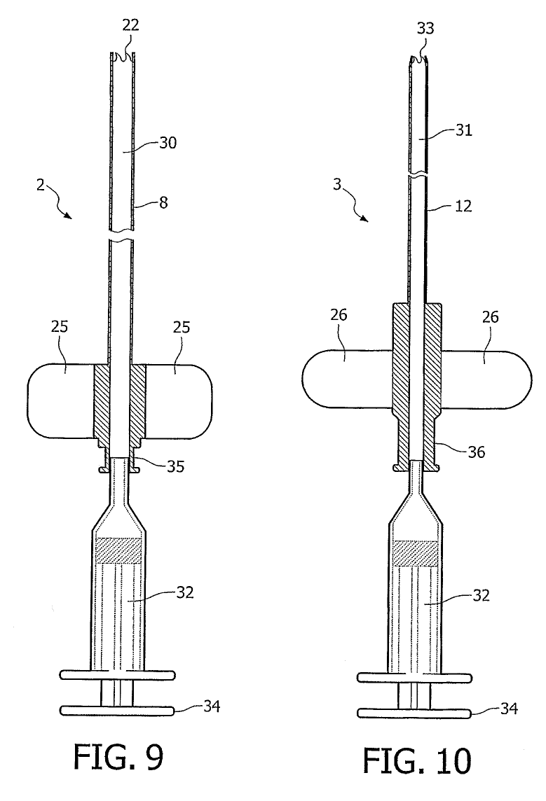

FIG. 9 illustrates an embodiment of an external trephine of a device according to the present invention on the proximal end of which a syringe has been fitted.

FIG. 10 illustrates an embodiment of an internal trephine of a device according to the present invention on the proximal end of which a syringe has been fitted.

### DETAILED DESCRIPTION

The present invention relates to a surgical device for making a bore in a tissue or joint and/or for delivering a medicament or a medical material to a tissue or joint. In the following description, the present invention will be described in terms of its use to access and/or treat bone tissues or joints. However, it will be clear that the present surgical device may also be used for making a bore and/or of delivering a medicament, or a medical material to any kind of tissue, joint or organ of a subject in need thereof.

According to the present invention the term “trephine” as used herein refers to a surgical instrument, which is generally used for harvesting bone plugs or for cutting holes in bones. Trephination, as used herein, denotes either removal of a bone section or simple drilling or boring of a hole in a bone or a bone tissue or a joint. A trephine may be used in accordance with the present invention for diagnostic purposes, e.g. for core biopsy, as well as for therapeutic applications, e.g. for the delivery of a medicament or material.

The terms “bore”, “hole” or “cavity”, are used herein as synonyms.

The terms “bone”, “bone tissue” or “osseous tissue” are used herein as synonyms and all refer to the connective tissue forming the skeleton of vertebrates, and consisting of bone-building osteoblasts, stationary osteocytes, and bone-destroying osteoclasts, embedded in a mineralized matrix infused with spaces and canals.

The term “joint” as used herein refers to a point of articulation between two or more bones, especially such a connection that allows motion. The term joint may refer to the hip, ankle, knee, elbow, wrist, shoulder.

The present surgical instrument is particularly suitable for making a bore in a bone tissue or joint and/or for treating, i.e. delivering medicaments or materials to said bone tissue or to said joint in order to treat bone diseases. The term “bone diseases” or “osseous diseases” are used herein as synonyms and refer to any type of bone disease, the treatment of which requires the formation of a bore in the bone tissue or a joint, and/or the delivery of a medicament or material to said tissue or joint. This term may include but is not limited to local bone diseases such as osteonecrosis, any type of fractures, osseous rebuilding in the not-union fractures (pseudarthrose), axillo-facial fractures, spinal fusions and rebuilding, etc. . . . and systemic diseases such as multi-site osteonecrosis, severe osteoporosis, etc.

The term “medicament” as used herein refers to any type of compound in any type of formulation, such as but not limited to a liquid form or a paste-like form, which may be applied to treat bone diseases. The term “medicament” as used herein may also encompass any type of compound in any type of formulation, such as but not limited to a liquid form or a paste-like form, which may be applied for diagnostic purposes.

The term “material” as used herein refers to any type of material, including synthetic or cellular material, which may be applied to treat diseases, preferably bone diseases. Non limitative examples of synthetic materials may include prostheses, anchors, implants, cellular material, etc. The term “cellular material” as used herein refers to cells that are used to treat diseases. Such cellular material is intended to replace, repair or improve the biological function of a tissue, joint or organ.

The term “subject” as used herein may refer to a human, although it will be appreciated by those in the art, that the subject may be animal as well. Animals, including mammals such as cats, dogs, farm animals including cows, horses, goats, sheep, pigs, etc., are included within the definition of subject.

### Surgical Device

In a first embodiment, the present invention provides a surgical device adapted for making a bore in a tissue or a joint of a subject, comprising at least two trephines.

The device comprises at least one external trephine, which is used in conjunction with an awl which is adapted to be positioned and to slide inside the external trephine. This element of the device is used for penetrating by hand in the tissue and to be inserted at the right place. A syringe may be couple to the external trephine, such that said trephine may act as an injection device and can be used to apply a medicament or material to the bore.

The device further comprises at least one internal trephine, adapted to be positioned and to slide inside said external trephine, and to be used in conjunction with a guiding rod, which is adapted to be positioned and to slide inside said internal trephine. The internal trephine may be used to perform a core biopsy, for instance to obtain bone marrow together with bone fibers. After the internal trephine is removed from the tissue, such solid sample may be pushed out of the trephine with the guiding rod and the sample may be examined, for instance under a microscope to see the cells and architecture of the bone marrow. Alternatively, the core biopsy may also be returned to bore in the bone tissue or joint is a similar way, i.e. by pushing the sample out of the trephine with the guiding rod, for instance after having applied a medicament or material to the bore. The returned sample may then cover the medicament or material that has been applied. Alternatively, or in combination therewith, the present device may thus comprises at least one internal trephine, for taking a core biopsy, and at least one additional internal trephine, which may be coupled to a syringe, such that such internal trephine may also act as an injection device.

In a preferred embodiment, the invention provides a surgical device adapted for making a bore in a tissue or a joint, wherein the external trephine and the internal trephine both comprise hollow tubular elements having an open distal end and an open proximal end. Preferably, the tubular portion of the trephines is circular in cross section, although the trephine may also adopt another cross-sectional configuration; and for instance be polygonal in cross section.

The present surgical device also comprises a guiding rod having dimensions that are adapted in order to enable the positioning of the rod inside the internal trephine and/or external trephine. The guiding rod preferably comprises a solid tubular portion of which the proximal end is provided with an abutment surface for limiting the movement of the guiding rod in distal direction, for instance when inserted in the internal and/or external trephine. The distal end of the rod may be blunt or rounded or have a conical shape. Preferably, the tubular portion of the guiding rod is circular in cross section, although the portion may have any cross sectional shape permitting insertion and axial sliding within an internal trephine; and for instance be polygonal in cross section.

The surgical device further comprises an awl having dimensions that are adapted in order to fit inside the external trephine. The awl preferably comprises a solid tubular portion of which the proximal end is provided with a handle for handling the instrument. Preferably, the tubular portion of the awl is circular in cross section, although the portion may have any cross sectional shape permitting insertion and axial sliding within an external trephine; and for instance be polygonal in cross section. In a preferred embodiment the distal end of the awl is provided with a cutting blade. Such cutting blade may be provided with at least two, and preferably at least three cutting edges, and is particularly suitable for penetrating a tissue and boring a hole in a tissue. The awl is used for penetrating in a bone tissue and to position the external trephine at the right place in the bone tissue.

In a preferred embodiment, the external and the internal trephine according to the present invention both have an open distal end which is provided with a cutting rim. In a preferred embodiment, the cutting rim of the internal and the external trephine is provided with cutting teeth, although in certain circumstances merely a sharpened edge may be present and sufficient for cutting. When present, the cutting teeth are according to a preferred embodiment curved, as illustrated on FIG. 3-4. The term “curved” refers to the fact that one lateral side of a tooth is longer than the opposite side of the tooth. In a preferred embodiment, the teeth of the internal trephine extend to the interior of the internal trephine; i.e. towards the lumen of the internal trephine, as illustrated on FIG. 4B. The number of teeth and their length may vary. In addition, the number of teeth and their length on the internal and the external trephine may be equal or different. In a particularly preferred embodiment, between 3 to 12 teeth may be present on the cutting rim of the external and of the internal trephine. In another preferred embodiment, the length of the teeth may be comprised between 0.3 and 4 mm, and preferably between 1.5 and 3 mm, and for instance between 2 or 3 mm.

In another preferred embodiment, the invention relates to a device wherein the proximal end of the external trephine and of the internal trephine is provided with an adaptor element. These adopter elements preferably comprise laterally extending wings for facilitating manual boring of the trephines in a tissue or joint. The wings of the adaptor elements of both internal and external trephines may be in the form of a T-piece, and act as an operating handle, which greatly facilitate handling of the trephine, and manual boring of the trephines in a tissue or joint. An important advantage of the present surgical device and of such elements is the possibility to insert the present trephines by hand through the skin and by a percutaneous approach, reducing the aggressiveness and improving the safety of the penetration method.

The adaptor element of the external trephine preferably further comprises a proximal abutment surface positioned proximally of the wings, which is capable of abutting against a distal abutment surface of the internal trephine or the handle of an awl, as illustrated of FIGS. 7 and 8, respectively. The adaptor element of the internal trephine preferably further comprises a distal abutment surface located distally of the wings for limiting movement of the internal trephine in distal direction when the internal trephine is inserted in the external trephine. This distal abutment surface serves to limit distal movement of the internal trephine when inserted in the external trephine by bearing against and abutting the proximal abutment surface of the adaptor element of the external trephine, as illustrated of FIG. 7. As illustrated on FIGS. 1b and 2b, the adaptor element of the internal trephine may further comprise a proximal abutment surface located proximally of the wings. Alternatively, such proximal abutment surface may be absent, as illustrated on FIG. 2e.

The adaptor element enables easy and rapid manual screwing of the trephine in bone tissue. In addition, the adaptor elements of internal and external trephine are suitable for coupling a syringe. As illustrated on FIG. 9, the adaptor element of an external trephine of the surgical device according to the invention is adapted to removably receive an injection syringe. In another embodiment, as illustrated in FIG. 10, the adaptor element of an internal trephine of the surgical device according to the invention is adapted to removably receive an injection syringe.

The length of the awl, the guiding rod, and the external and internal trephines may vary, in function of the bone tissue or joint to be reached. Preferably, the length of the internal trephine is greater than the length of the external trephine, and an extended portion of the internal trephine projects beyond the proximal end of the external trephine, when fully inserted herein, as illustrated on FIG. 7. Preferably, the length of the internal trephine is between 1.05 and 1.30 times greater than the length of the external trephine, and for instance 1.10; 1.12; 1.15; 1.17; 1.20; 1.22; 1.25; 1.27; or 1.30 times greater than the length of the external trephine.

In another preferred embodiment, the length of the guiding rod is greater than the length of the internal trephine, and an extended portion of the guiding rod projects beyond the proximal end of the internal trephine, when fully inserted herein, as illustrated on FIG. 7. Preferably, the length of the guiding rod is between 1.05 and 1.30 times greater than the length of the internal trephine, and for instance 1.10; 1.12; 1.15; 1.17; 1.20; 1.22; 1.25; 1.27; or 1.30 times greater than the length of the internal trephine.

In another preferred embodiment, the length of the awl is greater than the length of the external trephine, and an extended portion of the awl, preferably the portion containing the cutting blade or at least a part thereof, projects beyond the proximal end of the external trephine, when fully inserted herein, as illustrated on FIG. 8. Preferably, the length of the awl is between 1.05 and 1.30 times greater than the length of the external trephine, and for instance 1.10; 1.12; 1.15; 1.17; 1.20; 1.22; 1.25; 1.27; or 1.30 times greater than the length of the external trephine

In a preferred embodiment the external trephine has a length comprised between 30 and 300 mm, and preferably between 70 and 200 mm, and for instance of 90, 100, 110, 125, 135, or 150 mm. In another preferred embodiment, the internal trephine has a length comprised between 30 and 350 mm, and preferably between 80 and 300 mm, and for instance of 120, 150, 170, 190, 210, 230, 250, or 270 mm. In yet another preferred embodiment, the guiding rod has a length comprised between 35 and 350 mm, and preferably between 40 and 350 mm, and for instance of 50, 70, 90, 100, 125, 135, 150, 175, 200, 225, 250, 275, 300 or 325 mm. In yet another preferred embodiment, the awl has a length comprised between 35 and 350 mm, and preferably between 40 and 350 mm, and for instance of 50, 70, 90, 100, 125, 135, 150, 175, 200, 225, 250, 275, 300 or 325 mm.

The present surgical device is in particular characterized in that it comprise at least one internal and one external trephine, whereby the internal trephine is adapted to be positioned in close contact with the external trephine and capable of sliding therein. In accordance with the present invention, both trephines have a distal end which is provided with a cutting rim. Therefore, the present invention provides a device which enables to make two bores in a tissue or bone; an outer bore, of which the circumference corresponds to the outer diameter of the external trephine and an inner bore of which the circumference corresponds to the outer diameter of the internal trephine. The conical shape of the distal end of the internal external trephine advantageously enables to obtain a bore with neat edges and optimal dimensions, and to take a core biopsy, if required. Moreover, the internal trephine is somewhat longer than the external trephine.

In a preferred embodiment, the outer diameter of the external trephine is smaller than 10 mm, and preferably smaller than 8 mm, and preferably smaller than 6 mm, and for instance comprises 5, 4, 3 or 2 mm. In another preferred embodiment, the outer diameter of the internal trephine is smaller than 10 mm, and preferably smaller than 8 mm, and for instance comprises 5, 4, 3 or 2 mm. In yet another preferred embodiment, the guiding rod has a diameter that is smaller than 8 mm, and more preferably smaller than 5 mm, and more preferably smaller than 3 mm, and for instance comprises 1.5 or 2 mm. In yet another preferred embodiment, the awl has a diameter that is smaller than 10 mm, and more preferably smaller than 8 mm, and more preferably smaller than 5 mm, and for instance comprises 5, 4, 3 or 2 mm.

In yet another embodiment the invention provides a surgical device, wherein the diameter of the guiding rod is smaller than the inner diameter of the internal trephine and preferably 1.05 to 1.10 times smaller, and for instance 1.06; 1.07; 1.08; 1.09; or 1.10; times smaller than the inner diameter of the internal trephine, such that the guiding rod can slide in the internal trephine.

In a further embodiment the invention provides a surgical device, wherein the diameter of the awl is smaller than the inner diameter of the external trephine and preferably 1.05 to 1.10 times smaller, and for instance 1.06; 1.07; 1.08; 1.09; or 1.10; times smaller than the inner diameter of the external trephine, such that the awl can slide in the external trephine.

In one embodiment the invention provides a surgical device comprising an internal and an external trephine, wherein the outer diameter of the internal trephine is smaller than the inner diameter of the external trephine. In such embodiment, the outer diameter of the distal end of the internal trephine and the outer diameter of the tubular portion of the internal trephine are similar. Preferably, the invention provides a device wherein the outer diameter of the internal trephine is 1.05 to 1.25 times smaller, and preferably 1.05 to 1.10 times smaller than the inner diameter of the external trephine, and for instance 1.06; 1.08; 1.10; 1.12; 1.14; 1.16 or 1.18 times smaller than the inner diameter of the external trephine, such that the internal trephine is adapted to be positioned in close contact with the external trephine and capable of sliding therein. In such embodiment, it is possible to obtain by using the set of two trephines, a typical bore configuration whereby an inner bore of a smaller diameter is created inside and extends deeper than an outer bore. Such bores have an optimal configuration for optimally receiving—for instance liquid or paste-like—medicaments or materials in the excavated osseous tissues or joints. Medicaments or materials can be easily delivered to such bores; while limiting loss caused by out-flow out of the bore and contamination or infiltration of tissues surrounding the bore with the medicaments or materials.

In an alternative embodiment, the internal trephine has an outer diameter which is preferably similar to or larger than the outer diameter of the external trephine. In such embodiment, the invention provides a surgical device wherein the outer diameter of the distal end of the internal trephine is different form the outer diameter of the tubular portion of the internal trephine. In such embodiment the diameter of the distal end of the internal trephine is preferably expandable to a diameter which is equal to or larger than the outer diameter of the external trephine when the internal trephine is fully inserted in the external trephine and extends beyond the proximal end of the external trephine. Preferably, the outer diameter of the distal end of the internal trephine is shrinkable to a diameter which is equal to or smaller than the outer diameter of the internal trephine when the internal trephine is retracted in the lumen of the external trephine. In an example, an internal trephine is provided having an outer diameter (of the tubular portion) of 2 mm. The internal trephine is able to slide in an external trephine having an outer diameter of 4 mm. The internal trephine has a distal end which is able to adopt an outer diameter of 4 or 5 mm. This outer diameter will shrink to a diameter of 2 mm when the internal trephine is retracted in the lumen of the external trephine.

In use, when the distal part of the internal trephine is pushed out of the external trephine, it expends and bores a hole larger than the bore made by the external trephine. When the bore is made, the distal end of the internal trephine can be retracted inside the external trephine, eventually carrying a sample of bone tissue in its lumen. In such embodiment, it is possible to obtain by using the set of two trephines, a typical bore configuration whereby a bore having a diameter which is similar to or larger than the diameter created by the external trephine. Such bores have an optimal configuration for optimally taking bore samples of a suitable size.

The present invention further provides a surgical device, wherein the external trephine has a thickness comprised between 0.5 and 1.5 mm, and preferably between 0.7 and 1.2 mm. In yet another embodiment, the invention provides an internal trephine having a thickness comprised between 0.5 and 1.5 mm, and preferably between 0.7 and 1.2 mm.

The external and the internal trephine, the guiding rod and the awl are generally made from biocompatible materials, which are sterilisable, such as for instance hardened stainless steel.

In accordance with the present invention, the external and the internal trephine, the guiding rod and the awl may be packaged, for instance in a package or housing with recesses for awl, internal and external trephine, and the guiding rod.

Referring now to the figures, FIGS. 1 and 2 illustrate the different elements of an embodiment of surgical device 1 according to the present invention. FIG. 1a and 2a illustrates an embodiment of an external trephine 2. The trephine 2 has an open distal end 6, an open proximal end 7 and a hollow tubular portion 8, preferably of circular cross section, with an inner diameter sized to receive an internal trephine 3 and a guiding rod 4 within the lumen 30 of the external trephine 2. The distal end 6 of the trephine 2 is provided with a cutting rim 9. As illustrated on FIGS. 2a, 3A and 4A, this cutting rim 9 is provided with cutting teeth 22, which are curved. The proximal end 7 of the external trephine 2 is further provided with an adaptor element 23. The adaptor element 23 of the external trephine 2 is provided with laterally extending wings 25 for facilitating handling of the trephine and boring of the trephine in a tissue or joint. The adaptor element 23 further comprises proximally of the wings 25 a proximal abutment surface 35. The abutment surface 35 may be adapted for connecting a syringe type luer or luer lock. An advantageous feature of the external trephine of the surgical device according to the invention is thus that it is adapted to be removably coupled to an injection syringe 32, as illustrated for instance on FIG. 9.

FIGS. 1b, 2b and 2e illustrate embodiments of an internal trephine 3. The trephine 3 has an open distal end 10, an open proximal end 11 and a hollow tubular portion 12, which is preferably circular in cross section. The internal trephine preferably has an inner diameter sized to receive a guiding rod 4 within the lumen 31 of the internal trephine 3 and an outer diameter sized to be received within the lumen of the external trephine 30, as illustrated on FIG. 7. The distal end 10 of the trephine 3 is provided with a cutting rim 13. As illustrated on FIG. 2b, 2e, 4A-B, this cutting rim 13 is provided with cutting teeth 33, which are curved and extend to the interior of the trephine 3. The proximal end of the internal trephine 3 is further provided with an adaptor element 24. The adaptor element 24 of the internal trephine 3 is further provided with laterally extending wings 26 for facilitating handling of the trephine and manual boring of the trephine in a tissue or joint. The adaptor element 24 further comprises distally of the wings 26 a distal abutment surface 37 which serves to limit distal movement of the internal trephine 3 when inserted in the lumen 30 of the external trephine, as illustrated on FIG. 7, by bearing against and abutting the proximal abutment surface 35 of the adaptor element 23 of the external trephine 2. The adaptor element 24 may further comprises, proximally of the wings 26, a proximal abutment surface 36, as indicated in FIG. 2b. The abutment surface 36 may be adapted for connecting a syringe type luer or luer lock. An advantageous feature of the internal trephine of the surgical device according to the invention is that it is adapted to be removably coupled to an injection syringe 32, as illustrated for instance on FIG. 10.

In another preferred embodiment (not illustrated), the proximal abutment surface of the internal trephine has a configuration and an inner diameter equal to the proximal abutment surface of the external trephine, such that a same type and dimension of syringe may be used to inject a material through the lumen of the external or of the internal trephine. The inner diameter of the proximal abutment surface of the internal trephine may therefore be different from the inner diameter of the tubular portion of the internal trephine. Similarly, in yet another embodiment, the inner diameter of the proximal abutment surface of the external trephine may therefore be different from the inner diameter of the tubular portion of the external trephine.

FIGS. 1c and 2c illustrate an embodiment of a guiding rod 4. The guiding rod 4 is an elongate rigid rod, preferably of circular cross section with a diameter sized to be received within the lumen 31 of the internal trephine 3. The guiding rod 4 is terminated in its distal end with an essentially blunt end 29. It will be clear however that other configurations of the distal end of the guiding rod are also encompassed by the present invention. The proximal end 15 of the guiding rod 4 is provided with an abutment surface 17. This abutment surface 17 serves to limit distal movement of the guiding rod 4 when inserted in the internal trephine 3 as illustrated on FIG. 7 by bearing against and abutting the proximal abutment surface 36 (if present) of the adaptor element 24 of the internal trephine 3.

FIGS. 1d and 2d illustrate an embodiment of an awl 5. The awl 5 is an elongate rigid rod, preferably of circular cross section with a diameter sized to be received within the lumen 30 of the external trephine 3. In a preferred embodiment, the diameter of the awl 5 is equal to the outer diameter of the internal trephine 3. The proximal end 19 of the awl 5 is preferably provided with a handle 21 or the like adapted to easily hold the instrument. This handle may also function as an abutment surface, when the awl is fully inserted in the lumen of the external trephine. The awl has a distal end 18, which is provided with a cutting blade 38. When fully inserted in the external trephine, as illustrated on FIG. 8, the cutting blade 38 preferably extends longitudinally along the axis of the external trephine and has an end provided with two or more cutting edges forming a point in the plane of the distal opening 6 of the trephine 2. The blade 38 shown in FIG. 2d is at its end provided with three cuffing edges which may extend to the exterior of the trephine 2 when the awl is inserted herein, in order to guide the trephine 2, and more generally the surgical device, when boring in a tissue or joint.

FIG. 5 and FIG. 6 illustrate alternative embodiments of the distal end of an external trephine. It will be clear that the illustrated features may also be provided on the distal end of an internal trephine according to the present invention. As illustrated on FIG. 5, the distal end of the external trephine 2 is provided with holes 39. Such holes greatly facilitate application of a medicament or a material to bore that has been made in the bone tissue or joint. As illustrated on FIG. 6, the distal end of the external trephine 2 is further provided with retractable spines 40. Such retractable spines can advantageously be used to create small hole or cracks into the surrounding tissues and facilitating the diffusion of a medicament or material applied to the bone tissue or joint.

FIG. 7 illustrates how the different elements of a surgical device according to the invention are arranged and encased with respect to each other. Guiding rod 4 is introduced in the lumen of the internal trephine 3, which latter is introduced in the lumen of the external trephine 2. The length of the internal trephine 3 is greater than the length of the external trephine 3 such that an extended portion 27 of the internal trephine 3 projects beyond the proximal end of the external trephine 2, when fully inserted herein. In addition, the length of the guiding rod 4 is greater than the length of the internal trephine 3 such that an extended portion 28 of the guiding rod 4 projects beyond the proximal end 11 of the internal trephine 2, when fully inserted herein.

FIG. 8 illustrates how an external trephine and an awl of a surgical device according to the invention are arranged and encased with respect to each other. The awl 5 is introduced in the lumen of the external trephine 2. The length of the awl 5 is greater than the length of the external trephine 2, such that an extended portion 41 of the awl, comprising the cutting blade 38, projects beyond the proximal end of the external trephine 2, when fully inserted herein.

The trephines of the present invention may be coupled to a syringe, type luer or luer lock, and act as injection devices, as illustrated on FIGS. 9 and 10. The proximal end of the external or internal trephine of the surgical device is preferably adapted to be receive the syringe. FIG. 9 for instance illustrates an embodiment of an external trephine 2 on the proximal end of which a syringe 32 with piston 34 has been fitted. FIG. 10 illustrates an embodiment of an internal trephine 3 on the proximal end of which a syringe 32 with piston 34 has been fitted. In a preferred embodiment, the syringe may be pre-filled with a suitable medicament or material, for instance with cellular material, which is to be injected in the excavated tissue or joint of a subject in need thereof.

The present surgical device and/or the present kit may be used for making a bore in a tissue, such as a bone tissue, or a joint, for instance for performing a biopsy. To this end, at a trephination site is selected on the subject's body, and a small incision through is made the skin large enough to accept the external trephine of the present device, which is then inserted into the incision and depressed against the tissue or joint. In a first step, the awl is engaged in the lumen of the external trephine and awl and trephine are inserted percutaneously by hand into the selected site. The awl and the external trephine are introduced under radioscopy until the right area. The external trephine has teeth at its distal edge for securely gripping the surface of the tissue or joint. A trephine hole is drilled in the tissue or joint by passing the awl through the lumen in the external trephine and drilling the awl in the tissue or joint. Using the present elements of the surgical device, progression of the external trephine is easier because the tissue is advantageously crushed by the cutting blade (nail tip) of the awl. Tissue samples can be taken by the external trephine of the present device, e.g. in the lumen of the trephine, when the awl is removed. Then, the awl is removed and an internal trephine is introduced in the external trephine. A trephine hole of a limited length and a smaller or larger diameter (depending on the configuration of the distal end of the internal trephine) is now drilled in the tissue or joint by passing the internal trephine through the lumen in the external trephine and drilling the internal trephine in the bone tissue or joint. A tissue sample is taken up in the lumen of the internal trephine of the present device. This sample may be removed from the trephine by pushing the guiding rod in the lumen of the trephine. The tissue sample may for instance be removed from the subject's body for further examination and/or optionally replaced in the bore, e.g. by pushing the sample with the guiding rod out of the trephine back in the bore (see further below).

In another application the present surgical device or the present kit may also be used for delivering a medicament or material to a tissue, such as a bone tissue, or to a joint. Preferably, the present surgical device is used for delivering a medicament or material in the bore made in the tissue or joint. The present device may therefore be used for treating various bone diseases as defined above. To this end, a trephination hole is made by the method as indicated above, and a tissue sample is removed from the subject's body by the internal trephine. Subsequently, a syringe is mounted on the external trephine and injected in the trephination hole. Alternatively, a syringe may also be mounted on a second internal trephine, different from the one used to take a tissue sample out of the subject's body. Optionally, after having applied a medicament or material to the bore, the tissue sample taken from the subject's body using the first internal trephine may be returned to the body, by re-introducing the internal trephine in the lumen of the external trephine and by pushing a guiding rod through the lumen of the internal trephine. The returned sample may thus cover the medicament or material that has been applied to the bore.

A particular advantageous use of the present invention is for the delivery of cellular material to a tissue or joint. Cellular therapy may offer a biological solution in alternative to existing (non-biological) treatment methods. It may also provide a complementary or bonus effect to existing treatments, for example improvement of the life prediction or life quality, etc. . . . Cellular therapy may comprise a novel treatment method for human health problems for which to date no satisfactory treatments exist.

In the appended drawings following annotations were used.

| | |
|---|---|
| 1 | surgical device |
| 2 | external trephine |
| 3 | internal trephine |
| 4 | guiding rod |
| 5 | awl |
| 6 | distal end external trephine |
| 7 | proximal end external trephine |
| 8 | tubular portion external trephine |
| 9 | cutting rim external trephine |
| 10 | distal end internal trephine |
| 11 | proximal end internal trephine |
| 12 | tubular portion internal trephine |
| 13 | cutting rim internal trephine |
| 14 | distal end guiding rod |
| 15 | proximal end guiding rod |
| 16 | tubular portion guiding rod |
| 17 | abutment surface guiding rod |
| 18 | distal end awl |
| 19 | proximal end awl |
| 20 | tubular portion awl |
| 21 | handle awl |
| 22 | cutting teeth external trephine |
| 23 | adaptor element external trephine |
| 24 | adaptor element internal trephine |
| 25 | wings external trephine |
| 26 | wings internal trephine |
| 27 | an extended portion of the internal trephine |
| 28 | an extended portion of the guiding rod |
| 29 | Front end of guiding rod |
| 30 | lumen external trephine |
| 31 | lumen internal trephine |
| 32 | syringe |
| 33 | cutting teeth external trephine |
| 34 | Piston of syringe |
| 35 | proximal abutment surface external trephine |
| 36 | proximal abutment surface internal trephine |
| 37 | distal abutment surface internal trephine |
| 38 | cutting blade of awl |
| 39 | holes |
| 40 | retractable spines |
| 41 | an extended portion of the awl |

1. Surgical device adapted for making a bore in a tissue comprising:
an external trephine having an open distal end provided with a cutting rim, an open proximal end and a hollow tubular portion,
an internal trephine, adapted to be positioned and to slide inside said external trephine, and having an open distal end provided with a cutting rim, an open proximal end, and a hollow tubular portion, and
a guiding rod, which is adapted to be positioned and to slide inside said internal trephine.

2. Surgical device according to claim 1, further comprising an awl adapted to be positioned and to slide inside said external trephine having a distal end provided with a cutting blade, a proximal end provided with a handle and a solid tubular portion.

3. Surgical device according to claim 1, wherein the cutting rim of the external trephine and of the internal trephine is provided with cutting teeth.

4. Surgical device according to claim 1, wherein said teeth are curved.

5. Surgical device according to claim 1, wherein the distal end of the internal trephine has a conical shape.

6. Surgical device according to claim 1, wherein the teeth of the internal trephine extend to the interior of the internal trephine.

7. Surgical device according to claim 3, wherein said teeth have a length between 0.3 and 4 mm.

8. Surgical device according to claim 1, wherein the distal end of the external and/or internal trephine is provided with one or more hole.

9. Surgical device according to claim 1, wherein the distal end of the external and/or internal trephine is provided with retractable spines.

10. Surgical device according to claim 1, wherein the proximal end of the external trephine is provided with an adaptor element comprising laterally extending wings for facilitating manual boring of the trephine in a tissue or joint.

11. Surgical device according to claim 10, wherein the adaptor element of the external trephine further comprises a proximal abutment surface positioned proximally of the wings.

12. Surgical device according to claim 1, wherein the proximal end of the internal trephine is provided with an adaptor element comprising laterally extending wings for facilitating manual boring of the trephine in a tissue or joint.

13. Surgical device according to claim 12, wherein the adaptor element of the internal trephine further comprises a distal abutment surface positioned distally of the wings for limiting movement of the internal trephine in distal direction when inserted in the external trephine.

14. Surgical device according to claim 12, wherein the adaptor element of the internal trephine further comprises a proximal abutment surface positioned proximally of the wings.

15. Surgical device according to claim 1, wherein the guiding rod comprises a solid tubular portion of which the proximal end is provided with an abutment surface for limiting movement of the guiding rod in distal direction when inserted in the external and/or internal trephine.

16. Surgical device according to claim 1, wherein the cutting blade of the awl is provided with at least two cutting edges.

17. Surgical device according to claim 1, wherein the length of the internal trephine is greater than the length of the external trephine, and wherein an extended portion of the internal trephine projects beyond the proximal end of the external trephine, when the internal trephine is fully inserted in the external trephine.

18. Surgical device according to claim 17, wherein the length of the internal trephine is 1.05 to 1.30 times greater than the length of the external trephine.

19. Surgical device according to claim 1, wherein the length of the guiding rod is greater than the length of the internal trephine, and wherein an extended portion of the guiding rod projects beyond the proximal end of the internal trephine, when the guiding rod is fully inserted in the internal trephine.

20. Surgical device according to claim 19, wherein the length of the guiding rod is between 1.05 and 1.30 times greater than the length of the internal trephine.

21. Surgical device according to claim 1, wherein the length of the awl is greater than the length of the external trephine, and wherein an extended portion of the awl projects beyond the proximal end of the external trephine, when the awl is fully inserted in the external trephine.

22. Surgical device according to claim 21, wherein the length of the awl is between 1.05 and 1.30 times greater than the length of the external trephine.

23. Surgical device according to claim 1, wherein the diameter of the awl is 1.05 to 1.10 times smaller than the inner diameter of the external trephine.

24. Surgical device according to claim 1, wherein the diameter of the guiding rod is 1.05 to 1.10 times smaller than the inner diameter of the internal trephine.

25. Surgical device according to claim 1, wherein the outer diameter of the external trephine is smaller than 10 mm.

26. Surgical device according to claim 1, wherein the outer diameter of the internal trephine is 1.05 to 1.25 times smaller than the inner diameter of the external trephine.

27. Surgical device according to claim 1, wherein the outer diameter of the distal end of the internal trephine is different form the outer diameter of the tubular portion of the internal trephine, and wherein the diameter of the distal end of the internal trephine is expandable to a diameter which is equal to or larger than the outer diameter of the external trephine when the internal trephine is fully inserted in the external trephine and extends beyond the proximal end of the external trephine.

28. Surgical device according to claim 27, wherein the outer diameter of the distal ends of the internal trephine is shrinkable to a diameter which is equal to or smaller than the outer diameter of the internal trephine when the internal trephine is retracted in the lumen of the external trephine.

29. Kit comprising a surgical device comprising at least one internal trephine, external trephine, guiding rod, and optionally an awl according to claim 1, and a syringe for injecting a medicament or a material in a tissue or joint, whereby the syringe is adapted to be mounted on the internal and/or external trephine of the surgical device.

30. Kit according to claim 29, wherein said syringe is pre-filled with a medicament or a material.

31. A method for making a bore in a tissue with the surgical device of claim 2, comprising:
selecting a trephination site;
inserting the external trephine of claim 2 into an incision at the trephination site;
engaging the awl in the lumen of the external trephine and inserting the awl into the trephination site; and
drilling the tissue with the awl to make a bore.

32. The method of claim 31, further comprising delivering a medicament or material to the tissue through the bore.
